(19) United States
(12) Patent Application Publication
(10) Pub. No.: US 2025/0129374 A1
(43) Pub. Date: Apr. 24, 2025

(54) USE OF NON-A MODIFICATION OF POLY(A) TAIL IN PROMOTING MRNA TRANSLATION

(71) Applicant: INSTITUTE OF GENETICS AND DEVELOPMENTAL BIOLOGY, CHINESE ACADEMY OF SCIENCES, Beijing (CN)

(72) Inventors: Yusheng LIU, Beijing (CN); Falong LU, Beijing (CN)

(73) Assignee: INSTITUTE OF GENETICS AND DEVELOPMENTAL BIOLOGY, CHINESE ACADEMY OF SCIENCES, Beijing (CN)

(21) Appl. No.: 18/687,508

(22) PCT Filed: May 19, 2022
(86) PCT No.: PCT/CN2022/093735

§ 371 Date: Feb. 28, 2024

(30) Foreign Application Priority Data
Aug. 30, 2021 (CN) 202111002112.7

(51) Int. Cl. C12N 15/67; C12N 15/65

(52) U.S. Cl. CPC C12N 15/67; C12N 15/65; C12N 2830/50; C12N 2840/105

(57) ABSTRACT

Use of non-A modification of a poly(A) tail in promoting mRNA translation. It is found, by analyzing the poly(A) tails of human and mouse cells and mRNA translation, that the non-A modification of the poly(A) tail is positively correlated with the translation efficiency. Genetic analysis shows that GLD-4, an atypical poly(A) polymerase in a nematode, can regulate translation by establishing the poly(A) tail non-A modification. Furthermore, it has been proved by experiments that the non-A modification of a poly(A) tail can effectively promote mRNA translation, and the addition of G, C and U in the poly(A) tail can promote mRNA translation and improves the content of corresponding proteins. Therefore, it is proposed that the non-A modification of a poly(A) tail can be used as a new technical means for effectively promoting mRNA translation.

### CROSS REFERENCE TO RELATED APPLICATIONS

This is a US National Phase application based upon PCT Application No. PCT/CN2022/093735 filed May 19, 2022, which claims priority to Chinese Patent Application No. 202111002112.7 Aug. 30, 2021, and the disclosures of which are hereby incorporated by reference in their entirety.

### INCORPORATION OF SEQUENCE LISTING

The sequence listing that is contained in the file named “UNITP0126US_Sequence Listing_Amended_ST25”, which is 38 KB (as measured in Microsoft Windows®) and was created on Feb. 27, 2024, is filed herewith by electronic submission, and is incorporated by reference herein.

### FIELD

The present invention relates to use of a poly(A) tail with non-adenosine (non-A) modification in increasing mRNA translation in the field of biotechnology.

### BACKGROUND

Poly(A) tails affect RNA stability, nuclear export, and translation efficiency, and are thus critical for RNA metabolism. However, most transcriptome analysis loses poly(A) tail information due to the intrinsic defects in reading nucleic acid homopolymer sequences when using the next generation sequencing analysis platform. Therefore, the roles of RNA poly(A) tails in regulating the fate and functions of RNAs are urgently needed to be analyzed. Researchers have estimated the length of poly(A) tails on the Illumina platform using methods such as TAIL-seq and PAL-seq (poly(A)—TAIL length profiling by sequencing) (Chang et al., 2014; Subtelny et al., 2014). The translation efficiency of different genes is very different (Ingolia et al., 2009). Studies of RNA poly(A) tails in oocytes of various species, such as drosophila, xenopus and mice, have shown a clear correlation between the length of poly(A) tail and mRNA translation efficiency (Eichhorn et al., 2016; Lim et al., 2016; Luong et al., 2020; Subtelny et al., 2014; Yang et al., 2020). A longer poly(A) tail in oocytes can lead to more efficient translation of mRNA, but the same regulatory mechanisms do not exist in somatic cells (Luong et al., 2020; Park et al., 2016; Subtelny et al., 2014). Surprisingly, even a short poly(A) tail (shortest as 20 nt) can support translation of mRNA in somatic cells (Park et al., 2016). It has also been considered from some research point of views that it is enough as long as the length of poly(A) tail in somatic cells is longer than 20 nt, and that a longer poly(A) tail has no extra regulatory function on mRNA translation (Park et al., 2016).

TAIL-seq analysis results show that the 3′ end of RNA can incorporate other nucleotides through a terminal extension mechanism, and this modification is currently thought to regulate the stability of RNA, thus becoming another level of regulation of mRNA stability (Chang et al., 2014; Chang et al., 2018; Lim et al., 2014; Lim et al., 2018; Morgan et al., 2019; Morgan et al., 2017). The deadenylated short poly(A) tail may be modified with a terminal U modification by TUT4/7, which can tag and degrade the target RNA molecule (Lim et al., 2014). In contrast, a G modification introduced by TENT4A/B can prevent the deadenylation of the poly(A) tail by the CCR4-NOT complex, thereby increasing mRNA stability (Lim et al., 2018).

The nucleotide composition within the body of a poly(A) tail was largely unknown. Recently PAIso-seq and FLAM-seq methods based on the third generation sequencing platform were developed which are able to read long nucleic acid homopolymers such as a poly(A) tail (Legnini et al., 2019; Liu et al., 2019). Both methods detected a novel RNA modification that is widely distributed within the poly(A) tail of mouse oocytes, Caenorhabditis Elegans and human cell lines: non-adenosine (non-A) modification (poly(A) Tail Internal non-A Modifications, PATIM) (Legnini et al., 2019; Liu et al., 2019). However, its function is unknown. The proportion of transcripts containing pATIM varies greatly among different genes, which also raises an important scientific question: what function does pATIM have (Arango et al., 2018; Wang et al., 2015). The research on function of pATIM is of great significance.

### SUMMARY

The technical problem to be solved by the present invention is how to improve the efficiency of mRNA translation.

In order to solve the above technical problems, the present invention first provides any of the following uses:
1. Use of a poly(A) tail with a non-adenosine (non-A) modification incorporated in the poly(A) tail in mRNA translation or in increasing the efficiency of mRNA translation.
2. Use of a substance that promotes non-A modification in a poly(A) tail in increasing the efficiency of mRNA translation.
3. Use of a substance that promotes non-A modification in a poly(A) tail in producing a product for increasing the efficiency of mRNA translation.

The incorporation of a non-A modification into a poly(A) tail can be accomplished by direct chemical synthesis, biochemical methods, or by attaching a poly(A) tail containing non-A modification to the end of mRNA, or by transcribing an mRNA transcription template containing a non-A modified poly(A) tail.

Furthermore, the incorporation of a non-A modification into a poly(A) tail can be accomplished by biochemical methods out of a cell, or by introducing into a cell a gene encoding a protein with the function of promoting non-A modification in a poly(A) tail, that is, it can be done extracellularly or intracellularly.

The substances that promotes non-A modification in a poly(A) tail may be a substance that incorporates non-A modification into a poly(A) tail through chemical methods, or a substance that connects a poly(A) tail containing non-A modification to the end of a RNA that does not contain a poly(A) tail.

The substance that incorporates non-A modification in a poly(A) tail through biochemical methods may be a proteins, a gene, etc. that has the function of incorporating non-A modification into a poly(A) tail. The substance that can connect a poly(A) tail containing non-A modification to the end of a RNA that does not contain a poly(A) tail may be a protein, a gene, etc. that has the function of connecting a poly(A) tail containing non-A modification to the end of a RNA, or a composition comprising a protein, a gene, etc. that has the function of connecting a poly(A) tail containing non-A modification to the end of a RNA and a poly(A) tail containing non-A modification.

In the above uses, the transcription template may be a PCR double-stranded DNA product, an enzyme-digested double-stranded DNA product, a plasmid, a cosmid, a phage or a viral vector.

In the above uses, the non-A modification in the poly(A) tail may be selected from the group consisting of insertions of G, C, U and a combination thereof in the poly(A) tail, that is, the poly(A) tail containing non-A modification is a poly(A) tail containing not only A.

In the above uses, the substance that promotes non-A modification in the poly(A) tail is a protein or biomaterial, and the protein is selected from the group consisting of the following A1)-A5):
A1) a non-canonical PAP (ncPAP);
A2) GLD-4, TENT4A, or TENT4B protein;
A3) GLD-4 protein comprising an amino acid sequence set forth in SEQ ID NO: 2;
A4) a protein which is obtained by substituting and/or deleting and/or adding one or more amino acid residues of the amino acid sequence set forth in SEQ ID NO: 2 and has the same function; and
A5) a fusion protein obtained by connecting a label at the N end or/and the C end of A1) or A2) or A3) or A4); and
the biomaterial is selected from the group consisting of the following B1) to B5):
B1) a nucleic acid molecule encoding the above-mentioned proteins;
B2) an expression cassette comprising the nucleic acid molecule of B1);
B3) a recombinant vector comprising the nucleic acid molecule of B1), or a recombinant vector comprising the expression cassette of B2);
B4) a recombinant microorganism comprising the nucleic acid molecule of B1), or a recombinant microorganism comprising the expression cassette of B2), or a recombinant microorganism comprising the recombinant vector of B3); and
B5) a cell line comprising the nucleic acid molecule of B1), or a cell line comprising the expression cassette of B2).

In the above uses, the protein in A2) may be a protein that has 75% or higher identity with the GLD-4 protein and has the same function. The 75% or higher identity includes 75%, 80%, 85%, 90%, 95%, 96%, 97%, 98% or 99% identity.

In the above uses, the nucleic acid molecule in B1) may be selected from the group consisting of b11), b12), b13) and b14):
b11) a cDNA molecule or DNA molecule with a coding sequence of SEQ ID NO: 1;
b12) a DNA molecule set forth in SEQ ID NO: 1;
b13) a cDNA molecule or DNA molecule that has 75% or higher identity with the nucleotide sequence defined in b11) or b12) and encodes the protein;
b14) a cDNA molecule or DNA molecule that hybridizes to the nucleotide sequence defined in b11) or b12) or b13) under stringent conditions and encodes the proteins,
wherein, the nucleic acid molecules may be DNA, such as cDNA, genomic DNA or recombinant DNA; the nucleic acid molecules may also be RNA, such as mRNA or hnRNA.

Those of ordinary skill in the art can easily mutate the nucleotide sequences encoding the proteins of the present invention using known methods, such as directed evolution and point mutation. Those artificially modified nucleotide sequences that have 75% or higher identity with the nucleotide sequence of the proteins of the present invention are derived from the present invention and are equivalent to the sequences of the present invention, as long as the encoded proteins have a desired function.

The term “identity” as used herein refers to sequence similarity to a native nucleic acid sequence. “Identity” includes having 75% or higher, or 85% or higher, or 90% or higher, or 95% or higher identity with the nucleotide sequence encoding the protein consisting of the amino acid sequence set forth in SEQ ID NO: 2 of the present invention. Identity can be assessed with the naked eye or with computer software. Using computer software, the identity between two or more sequences can be expressed as a percentage (%), which can be used to evaluate the identity between related sequences.

In the above uses, the stringent conditions may be defined as follows: hybridization is performed in a mixed solution of 7% sodium dodecyl sulfate (SDS), 0.5 M NaPO₄ and 1 mM EDTA at 50° C., and washing is performed with 2×SSC and 0.1% SDS at 50° C. Alternatively, hybridization is performed in a mixed solution of 7% SDS, 0.5 M NaPO₄ and 1 mM EDTA at 50° C., and washing is performed with 1×SSC and 0.1% SDS at 50° C. Alternatively, hybridization is performed in a mixed solution of 7% SDS, 0.5 M NaPO₄ and 1 mM EDTA at 50° C., and washing is performed with 0.5×SSC and 0.1% SDS at 50° C. Alternatively, hybridization is performed in a mixed solution of 7% SDS, 0.5 M NaPO₄ and 1 mM EDTA at 50° C., and washing is performed with 0.1×SSC and 0.1% SDS at 50° C. Alternatively, hybridization is performed in a mixed solution of 7% SDS, 0.5 M NaPO₄ and 1 mM EDTA at 50° C., and washing is performed with 0.1×SSC and 0.1% SDS at 65° C. Alternatively, hybridization is performed in a mixed solution of 6×SSC and 0.5% SDS at 65° C., and the membrane is washed once with 2×SSC and 0.1% SDS, and once with 1×SSC and 0.1% SDS. Alternatively, hybridization is performed in a solution of 2×SSC and 0.1% SDS at 68° C., the membrane is washed twice, 5 minutes each time, then hybridization is performed in a solution of 0.5×SSC and 0.1% SDS at 68° C., and the membrane is washed twice, 15 minutes each time. Alternatively, hybridization and membrane washing are performed in a solution of 0.1×SSPE (or 0.1×SSC) and 0.1% SDS at 65° C.

The above-mentioned 75% or higher identity may be 80%, 85%, 90% or above 95% identity.

In the above uses, the expression cassette (gene expression cassette) containing the nucleic acid molecule encoding the protein of B2) refers to DNA that can express the protein in a host cell. The DNA includes not only a promoter for transcription of the gene encoding the protein, but also a terminator for transcription of the gene encoding the protein. Furthermore, the expression cassette may also include an enhancer sequence.

A recombinant vector containing the expression cassette of the gene encoding the protein may be constructed using an existing expression vector.

In the above uses, the vector may be selected from a group consisting of a plasmid, a cosmid, a phage and a viral vector.

In the above uses, the microorganism may be selected from a group consisting of yeast, bacteria, algae and fungi.

In the above uses, the cell line may or may not include propagation material.

In the above uses, the mRNA translation may be mRNA translation in a eukaryote.

In the above uses, the mRNA translation may be mRNA translation in a eukaryotic germ cell or somatic cell.

In the above uses, the eukaryote may be an animal or a plant or a eukaryotic microorganism.

The animal may be a mammal (such as a human, mouse, rabbit or pig) or a non-mammal (such as a zebrafish, fruit fly or Caenorhabditis Elegans).

The plant may be a dicotyledonous plant (such as Arabidopsis thaliana) or a monocotyledonous plant (such as rice).

The eukaryotic microorganism may be yeast.

The present invention further provides a method for increasing the efficiency of mRNA translation, comprising: performing non-A modification on the poly(A) tail of a target mRNA to increase the efficiency of the target mRNA translation.

In the above methods, the non-A modification of the poly(A) tail of the target mRNA can be completed by incorporating the non-A modification through direct chemical synthesis, or by generating a poly(A) tail containing non-A modification at the end of the mRNA through biochemical methods, or by connecting a poly(A) tail containing non-A modification to the end of the mRNA, or by transcribing an mRNA transcription template containing a poly(A) tail template with non-A modification.

In the above methods, the non-A modification may be achieved by applying the substance that promotes non-A modification of a poly(A) tail.

In the above methods, the mRNA translation may be mRNA translation in a eukaryote.

In the above methods, the mRNA translation may be mRNA translation in a eukaryotic germ cell or somatic cell.

In the above methods, the eukaryote may be an animal or a plant or a eukaryotic microorganism.

The animal may be a mammal (such as a human, mouse, rabbit or pig) or a non-mammal (such as a zebrafish, fruit fly or Caenorhabditis Elegans).

The plant may be a dicotyledonous plant (such as Arabidopsis thaliana) or a monocotyledonous plant (such as rice).

The eukaryotic microorganism may be yeast.

The present invention further provides a product for increasing the efficiency of mRNA translation, wherein the product is a substance that promotes non-A modification of a poly(A) tail.

In the above product, the mRNA translation may be mRNA translation in a eukaryote.

In the above product, the mRNA translation may be mRNA translation in a eukaryotic germ cell or somatic cell.

In the above product, the eukaryote may be an animal or a plant or a eukaryotic microorganism.

The animal may be a mammal (such as a human, mouse, rabbit or pig) or a non-mammal (such as a zebrafish, fruit fly or Caenorhabditis Elegans).

The plant may be a dicotyledonous plant (such as Arabidopsis thaliana) or a monocotyledonous plant (such as rice).

The eukaryotic microorganism may be yeast.

In the present invention, the non-A modification of a poly(A) tail or poly(A) tail with non-A modification refers to a poly(A) tail also containing G, C and/or U. The non-A modification of a poly(A) tail may be achieved by replacing any one or more A in the poly(A) tail with G, C and/or U, or by adding one or more G C and/or U at any position of the poly(A) tail. “More” refers to two or three or four or five or six or seven or eight or nine or ten, etc.

In the present invention, the position and number of non-A modification of G, C and/or U in the poly(A) tail are not particularly limited, which are all within the protection scope of the present invention as long as the translation efficiency can be improved.

In an embodiment of the present invention, poly(A) tails with non-A modification comprise sequences selected from SEQ ID NOs. 4, 5, and 6.

### BRIEF DESCRIPTION OF DRAWINGS

FIG. 1 is a flow chart of analysis of poly(A) tail sequences.

FIG. 2 shows that the non-A modification in poly(A) tail is positively correlated with translation efficiency. (A-D) Cumulative distribution frequency (CDF) of mRNA-normalized translational efficiency (TE) of poly(A) tails with/without non-A modification across the whole genome in different samples. The samples of the source of the plotted data were: (FIG. 2A) mouse oocytes at GV stage (number of genes: with G, n=4104; without G, n=1363; with C, n=4109; without C, n=1358; with U, n=4116; without U, n=1351); (FIG. 2B) Caenorhabditis Elegans germ cells (number of genes: with G, n=1390; without G, n=1585; with C, n=2233; without C, n=742; with U, n=2170; without U, n=805); (FIG. 2C) Caenorhabditis Elegans somatic cells (number of genes: with G, n=539; without G, n=934; with C, n=2323; without C, n=564; with U, n=909; without U, n=631); (FIG. 2D) HeLa cells (number of genes: with G, n=2075; without G, n=810; with C, n=2323; without C, n=562; with U, n=1884; without U, n=1001). The correlation between the non-A modification of the poly(A) tail and TE was calculated using the Kolmogorov-Smirnov (KS) test for p-value. (E-F) Quantitative protein abundance cumulative distribution frequency of gene with/without non-A modification of poly(A) tails by mass spectrometry. Data are obtained from GV stage oocytes (FIG. 2E) (number of genes: with G, n=2307; without G, n=768; with C, n=2308; without C, n=767; with U, n=2306; without U, n=769), and HeLa cells (FIG. 2F) (number of genes: with G, n=2398; without G, n=1029; with C, n=2694; without C, n=733; with U, n=2166; without U, n=1261), respectively. The p-value was determined by KS testing.

With G: pATIM-G, i.e., a poly(A) tail contains G.

Without G: A poly(A) tail does not contain G.

With C: pATIM-C, i.e., a poly(A) tail contains C.

Without C: A poly(A) tail does not contain C.

With U: pATIM-U, i.e., a poly(A) tail contains U.

Without U: A poly(A) tail does not contain U.

TE: Translational efficiency.

P-value is the KS statistical test P-value for the statistics of the two cumulative distribution curves.

FIG. 3 shows a sequence analysis of proteins encoded by 10 species of GLD-4 homologous genes. Underlining indicates amino acids important for catalytic activity and NTP binding.

FIG. 4 shows that GLD-4 is indispensable for the establishment of non-A modification of the poly(A) tail and the regulation of translational efficiency in Caenorhabditis Elegans. (FIG. 4A) Boxplots show the transcript ratios of each gene containing modifications of pATIM-G (left), PATIM-C(center) and pATIM-U (right) under the background of WT and gld-4 (ef15) mutant, respectively. Genes in the WT were divided into 2 groups (number of genes: low G, n=2758; high G, n=169; low C, n=2374; high C, n=553; low U, n=2392; high U, n=535) depending on whether the transcript contained a low proportion of non-A modifications in the poly(A) tail (proportion of the number of transcripts in WT containing G, C, and U modification in the number of total transcripts of poly(A) tails is less than 5%) or high (proportion of the number of transcripts in WT containing G, C, and U modification in the number of total transcripts of poly(A) tails is geater than or equal to 5%). The p-value was determined by two-tailed t-test. (FIG. 4B) Cumulative frequency distribution of mRNA-normalized ribosome binding (TE) changes in gld-4 RNAi (gld-4 mutants) and WT Caenorhabditis Elegans. The data show the gene with/without non-A modification in poly(A) tails (number of genes: with G, n=970; without G, n=2869; with C, n=1873; without C, n=1966; with U, n=1816; without U, n=2023), respectively. P-value is the KS statistical test P-value for the statistics of the two cumulative distribution curves.

FIG. 5 shows that non-A modification in poly(A) tail enhances translation efficiency. (FIG. 5A) A schematic graph of reporter mRNA with identical tail lengths but with different non-A modifications in poly(A) tails. (FIG. 5B) A schematic graph of a reporter system for the translation efficiency of the transcripts with different poly(A) tails. (FIG. 5C) Images of HeLa cells after 18 hours of transfection with reporter mRNA with different poly(A) tails. Length of the scale: 100 μm. DIC stands for differential interference versus bright field imaging. (FIG. 5D) Quantification results of expression by HeLa cells after 42 hours of transfection of reporter mRNA according to Fluorescence-Activated Cell Sorting (FACS). The upper graph is a fluorescence signal scattergram, and the lower graph is a fluorescence signal distribution histogram. (FIG. 5E) Quantification results of expression by Hela cells after 42 hours of transfection of reporter mRNA according to qRT-PCR (left, mRNA level) and immunoblotting (right, protein level, Flag tag fusion-expressed by all reporter genes is detected using anti-Flag antibody).

FIG. 6 shows quantitative analysis of the expression of reporter mRNA at different time points. (FIG. 6A) Quantitative analysis of the expression level of the reporter EGFP mRNA in HeLa cells at different time points after transfection as detected by FACS. The control group was cells that were not transfected with mRNA. A98 and A98pATIMs-2 were cells transfected with the reporter mRNAs containing A98 and A98pATIMs-2 tails, respectively. (FIG. 6B) Quantitative analysis of the relative fluorescence intensity of EGFP in Hela cells at different time points after transfection as detected by FACS. The cells involved in the analysis were the same as those in (FIG. 6A) above. P2 represents a threshold gate of low stringency, including cells with weak fluoresce; P3 represents a threshold gate of high stringency, including only cells with strong fluorescent.

### DETAILED DESCRIPTION

The present invention is described in further detail below with reference to specific examples, which are provided only for illustrating the present invention and not for limiting the scope of the present invention. The examples provided below serve as a guide for further modifications by a person skilled in the art and do not constitute a limitation of the present invention in any manner.

The experimental methods in the following examples, unless otherwise specified, were carried out in a conventional manner according to the techniques or conditions described in the literature in the art or according to the product instructions. Materials, reagents, instruments and the like used in the following examples are commercially available unless otherwise specified. In the quantitative experiments in the following examples, three replicates were set up and the average was taken as result. In the following examples, the first position of each nucleotide sequence in the sequence listing is the 5′ terminal nucleotide of the corresponding DNA/RNA, and the last position is the 3′ terminal nucleotide of the corresponding DNA/RNA, unless otherwise specified.

In the following examples, for the gld-4 (ef15) mutant (Mark Schmid, BeateKüchler, Christian R. Eckmann, Two conserved regulatory cytoplasmic poly(A) polymerases, GLD-4 and GLD-2, regulate meiotic progression in C. elegans, Genes & Dev. 2009. 23:824-836), the public can obtain the biological material from the applicant. The biological material is only used to repeat the relevant experiments of the present invention and cannot be used for other purposes. The gld-4 (ef15) mutant differs from background only in mutations of GLD-4 gene. The sequence of GLD-4 gene is shown in SEQ ID NO: 1, which encodes a GLD-4 protein shown in SEQ ID NO: 2.

### Example 1 Non-A Modification in Poly(A) Tail is a Conserved Characteristic of Poly(A) Tail in Eukaryotic RNAs

In this example, by analyzing poly(A) tails from human, mouse, rabbit, pig, zebrafish, fruit fly, Caenorhabditis Elegans, Arabidopsis thaliana, rice and yeast, it was found that non-A modification in poly(A) tail (abbreviated as pATIM) is a conserved characteristic of RNA poly(A) tails from eukaryotes, and its distribution is not species- or tissue-specific.

Samples to be tested: human Hela cells, mouse liver, rabbit liver, pig liver, zebrafish individual, fruit fly individual, Caenorhabditis Elegans individual, Arabidopsis thaliana, rice leaves, and yeast.

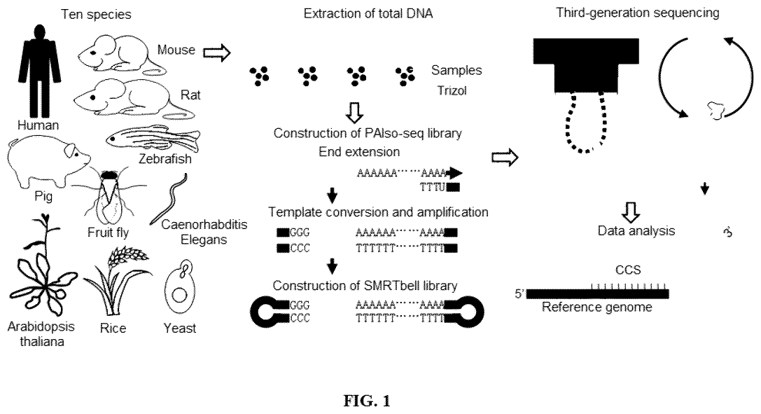

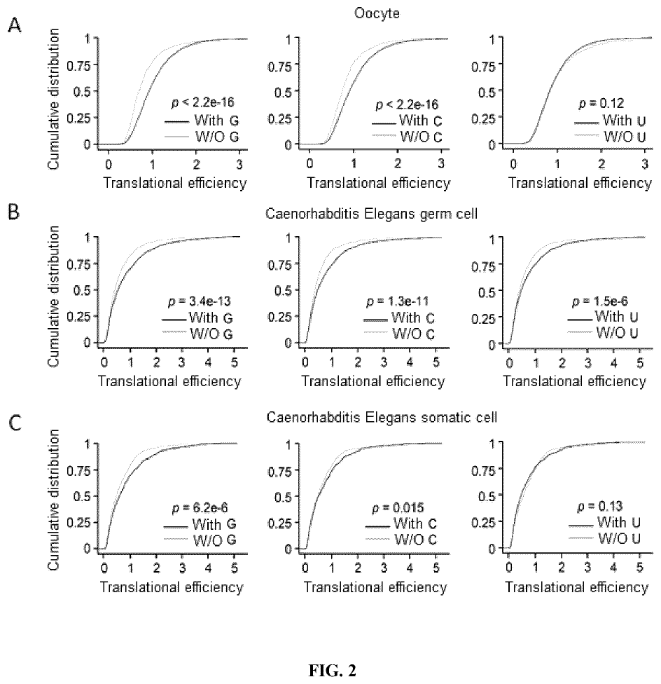

Total RNA of each sample was extracted, a PAIso-seq library was prepared, then third generation sequencing was carried out and the sequencing results were analyzed. The PAIso-seq sequencing sequences were aligned to the genome, and the sequences, which were at the end of RNA, not transcribed from a genomic template and cannot be aligned to the genome, were namely poly(A) tails. The detailed analysis method is disclosed in the literature (10.1038/s41467-019-13228-9). The steps are shown in FIG. 1, and examples of poly(A) tails of each species are shown in Table 1.

TABLE 1
Non-A modifications in poly(A) tails of 10 species

| | Sequencing | Gene | Number | Number | Number | Number | poly(A) tail sequence |
|---|---|---|---|---|---|---|---|
| Sample | ID | name | of A | of U | of C | of G | (5'->3') |
| HeLa cell | 71369484 | SLC3A2 | 91 | 1 | 2 | 1 | AAAAAAAAAAAAAAAAAAAAAAAA |
| | | | | | | | UGAAAAAAAAAAAAAAAAAAAAAA |
| | | | | | | | AAAAAACAAAAAAAAAAAAACAAA |
| | | | | | | | AAAAAAAAAAAAAAAAAAAAAAA |
| | | | | | | | (SEQ ID NO: 9) |
| HeLa cell | 65732695 | TSPAN6 | 82 | 1 | 1 | 1 | AAAAAAAAAAAAAAAAAAAAAAAA |
| | | | | | | | AAAAAAAAUGAAAAAAAAAAAAAA |
| | | | | | | | AAAAAAAAAAAAACAAAAAAAAAA |
| | | | | | | | AAAAAAAAAAAAA |
| | | | | | | | (SEQ ID NO: 10) |
| HeLa cell | 52626024 | NOL9 | 61 | 1 | 1 | 1 | AAAAAAAAAAAAAAAAAAAAAACA |
| | | | | | | | AAAAAAGAAUAAAAAAAAAAAAAA |
| | | | | | | | AAAAAAAAAAAAAAAA |
| | | | | | | | (SEQ ID NO: 11) |
| Mouse | 56296381 | Apoa2 | 91 | 1 | 1 | 1 | AAAAAAAAAAAAAAAAAAAAAAAA |
| liver | | | | | | | AAAAAAAAAAGAAAAAAACUAAAA |
| | | | | | | | AAAAAAAAAAAAAAAAAAAAAAAA |
| | | | | | | | AAAAAAAAAAAAAAAAAAAAAA |
| | | | | | | | (SEQ ID NO: 12) |
| Mouse | 43385682 | Cyp3all | 82 | 1 | 1 | 1 | AAAAAAUCAAAAAAAAAAAAAAAA |
| liver | | | | | | | AAAAAAAAAAAAGAAAAAAAAAAA |
| | | | | | | | AAAAAAAAAAAAAAAAAAAAAAAA |
| | | | | | | | AAAAAAAAAAAAA |
| | | | | | | | (SEQ ID NO: 13) |
| Mouse | 36373124 | Lrrfip1 | 40 | 5 | 2 | 1 | AAAAAAAACAUUUUAAAAAUGAAA |
| liver | | | | | | | AAAAAAAAAAACAAAAAAAAAAAA |
| | | | | | | | (SEQ ID NO: 14) |
| Rat liver | 31916121 | Itih3 | 132 | 1 | 1 | 2 | AAAAGCAGAAAAAAAAAAAAAAAA |
| | | | | | | | AAAAAAAAAAAAAAAAAAAAAAAA |
| | | | | | | | AAAAAAAAAUAAAAAAAAAAAAAA |
| | | | | | | | AAAAAAAAAAAAAAAAAAAAAAAA |
| | | | | | | | AAAAAAAAAAAAAAAAAAAAAAAA |
| | | | | | | | AAAAAAAAAAAAAAAA |
| | | | | | | | (SEQ ID NO: 15) |
| Rat liver | 5177533 | Psme1 | 103 | 1 | 1 | 1 | AAAAAAAAAAAAAAAAAAAAAAAA |
| | | | | | | | AAAAAUAAAAAAAAAAAAAAAAAA |
| | | | | | | | AAAAAAAAAAAAAAAAAAAAAAAA |
| | | | | | | | AAAAAAAAAACAAAAAAAAAAGAA |
| | | | | | | | AAAAAAAAAA |
| | | | | | | | (SEQ ID NO: 16) |
| Rat liver | 37421162 | Dpt | 93 | 2 | 2 | 1 | GUACCUAAAAAAAAAAAAAAAAAA |
| | | | | | | | AAAAAAAAAAAAAAAAAAAAAAAA |
| | | | | | | | AAAAAAAAAAAAAAAAAAAAAAAA |
| | | | | | | | AAAAAAAAAAAAAAAAAAAAAAAA |
| | | | | | | | AA |
| | | | | | | | (SEQ ID NO: 17) |
| Pig liver | 65274193 | AHSG | 107 | 1 | 1 | 1 | AAAAAAAAAAAAAAAAAAAAAAAA |
| | | | | | | | AAAAAAAAAUAAACAAAAAAAAAA |
| | | | | | | | AAAAAAAAAAAAGAAAAAAAAAAA |
| | | | | | | | AAAAAAAAAAAAAAAAAAAAAAAA |
| | | | | | | | AAAAAAAAAAAAAA |
| | | | | | | | (SEQ ID NO: 18) |
| Pig liver | 55313378 | DEF8 | 56 | 1 | 1 | 1 | AAAAAAAAAAAAAAGAAAAAAAAA |
| | | | | | | | AAAAAAAAAAAAAAAAAAUAAAAC |
| | | | | | | | AAAAAAAAAAA |
| | | | | | | | (SEQ ID NO: 19) |
| Pig liver | 12648941 | GTF2H3 | 55 | 1 | 2 | 1 | AAAAAACGUCAAAAAAAAAAAAAA |
| | | | | | | | AAAAAAAAAAAAAAAAAAAAAAAA |
| | | | | | | | AAAAAAAAAAA |
| | | | | | | | (SEQ ID NO: 20) |
| Zebrafish | 74252871 | cldnd | 157 | 1 | 1 | 1 | AAAAAAAAAAAAAAAAAAAAAAAA |
| | | | | | | | AAAAAAAAAAAAAAAAAAAAAAAA |
| | | | | | | | AAAAAAAAAAAAAAAAAAAAAAAA |
| | | | | | | | AAAAAAAAAAACAAAAAAAAAAAA |
| | | | | | | | AAAAAAAAAAAAAAAAAAGAAAAA |
| | | | | | | | AAAAAAAAAAAAAAAAAUAAAAAA |
| | | | | | | | AAAAAAAAAAAAAAAA |
| | | | | | | | (SEQ ID NO: 21) |
| Zebrafish | 29033213 | zp3c | 155 | 1 | 2 | 3 | AAAAAAAAAAAAAAAAAAAAAAAA |
| | | | | | | | AAAAAAAAAACAAAAAAAAAAAAA |
| | | | | | | | AAAAAAAAAAAAAGAAAAAAAAAA |
| | | | | | | | AAAAAUAAAAAAAAAAACAGAAAA |
| | | | | | | | AAAAAAAAAAAAAAAAAAAAAAAA |
| | | | | | | | AAGAAAAAAAAAAAAAAAAAAAAA |
| | | | | | | | AAAAAAAAAAAAAAAAA |
| | | | | | | | (SEQ ID NO: 22) |
| Zebrafish | 18023293 | emc2 | 138 | 2 | 1 | 2 | AAAAAAAAAAAACAAAAAAAAAAA |
| | | | | | | | AAAAAAAAAAAAAAAAAAAAAAAA |
| | | | | | | | AUUAAAAAAAAAAGAAAAAAAAAA |
| | | | | | | | AAAAAAAAAAAAAAAAAAAAAAAA |
| | | | | | | | AAAAAAAAAAAAAAAAAAAAAAAA |
| | | | | | | | AGAAAAAAAAAAAAAAAAAAAAA |
| | | | | | | | (SEQ ID NO: 23) |
| Fruit fly | 22151681 | dhd | 94 | 1 | 1 | 1 | AAAAAAAAAAAGAAAAAAAAAAAA |
| | | | | | | | AAAAAAAAAAAAAAAAAAAAAAAA |
| | | | | | | | AAAAAAAAAAAAAACAAAAAAAAA |
| | | | | | | | AAAAAAAAAAAAUAAAAAAAAAAA |
| | | | | | | | A |
| | | | | | | | (SEQ ID NO: 24) |
| Fruit fly | 63898007 | XNP | 93 | 1 | 1 | 1 | AAAAAAAAAAAAAAUAAAAAAAAA |
| | | | | | | | GAAAAAAAAAAAAAAAAAAAAAAA |
| | | | | | | | AAAAAAAAAAAAAAAAAAAAAAAA |
| | | | | | | | AAAAAAAAAAAAAAAAAAAAAACA |
| | | | | | | | (SEQ ID NO: 25) |
| Fruit fly | 30540381 | CG3275 | 59 | 2 | 1 | 1 | AAAAAAAAAAAAAAAAAAGTAAAA |
| | | 6 | | | | | AAAAAAAAAAACAAATAAAAAAAAA |
| | | | | | | | AAAAAAAAAAAAAA |
| | | | | | | | (SEQ ID NO: 26) |
| Caenorhabditis | 6292139 | wdfy-2 | 76 | 9 | 1 | 1 | AAAAAAAAAAAUCUUUUUUUUGAA |
| Elegans | | | | | | | AAAAAAAAAAAAAAAAAAAAAAAA |
| | | | | | | | AAAAAAAAAAAAAAAAAAAAAAAA |
| | | | | | | | AAAAAAAAAAAAAAA |
| | | | | | | | (SEQ ID NO: 27) |
| Caenorhabditis | 25428920 | rbm-28 | 45 | 10 | 1 | 1 | AAAAAAAAUUAUUUUUGUUUAAAA |
| Elegans | | | | | | | ACAAAAAAAAAAAAAAAAAAAAAA |
| | | | | | | | AAAAAAAAA |
| | | | | | | | (SEQ ID NO: 28) |
| Arabidopsis | 7602245 | PTAC16 | 94 | 1 | 0 | 1 | AAAAAAAAAAAAAAAAAAAAAAAA |
| thaliana | | | | | | | AAAAAAAAAAAAAAAAAAAAAAAA |
| | | | | | | | AAAAAAAAAAAAAAAAAAGUAAAA |
| | | | | | | | AAAAAAAAAAAAAAAAAAAAAAAA |
| | | | | | | | (SEQ ID NO: 29) |
| Arabidopsis | 74383463 | LHB1B | 68 | 1 | 0 | 2 | AAAAAAAAAAAAAAAAAAAAAAAA |
| thaliana | | 2 | | | | | AAAAAAUAAAGGAAAAAAAAAAAA |
| | | | | | | | AAAAAAAAAAAAAAAAAAAAAAA |
| | | | | | | | (SEQ ID NO: 30) |
| Arabidopsis | 13828929 | RPS29C | 63 | 1 | 1 | 0 | AAAAAAAAAAAAAAAAACAAAAAA |
| thaliana | | | | | | | AAAAAAAAUAAAAAAAAAAAAAAA |
| | | | | | | | AAAAAAAAAAAAAAAAA |
| | | | | | | | (SEQ ID NO: 31) |
| Rice | 5505833 | OsBBX | 51 | 0 | 0 | 1 | AAAAAAAAAAAAAAAAAAAAAAAA |
| | | 20 | | | | | AAAAAAAAGAAAAAAAAAAAAAAA |
| | | | | | | | AAAA |
| | | | | | | | (SEQ ID NO: 32) |
| Rice | 48562747 | PsaK | 87 | 2 | 1 | 1 | UUAACAAAAAAAAAAAAAAAAAAA |
| | | | | | | | AAAAAAAAAAAAAAAAAAAAAAAA |
| | | | | | | | AAAAAAAAAAAAAAAAAAAAAAAA |
| | | | | | | | AAAAAAAAAAGAAAAAAAA |
| | | | | | | | (SEQ ID NO: 33) |
| Rice | 6685582 | GRP162 | 44 | 0 | 1 | 1 | AAAAAAAAAAAAAAAAAAAAGAAA |
| | | | | | | | AAAAAAAAAAAAAAAAAAACAA |
| | | | | | | | (SEQ ID NO: 34) |
| Yeast | 34210566 | bg12 | 53 | 1 | 1 | 1 | AAAAAAAAAAGAAAAAAAAAAAAA |
| | | | | | | | AAAAAAAAAUCAAAAAAAAAAAAA |
| | | | | | | | AAAAAAAA |
| | | | | | | | (SEQ ID NO: 35) |
| Yeast | 67503084 | hsp150 | 51 | 1 | 2 | 2 | AAAAGGUAAAAAAAAAAAAAAAAA |
| | | | | | | | AAAAAAAAAAAAACAAAAAACAAA |
| | | | | | | | AAAAAAAA |
| | | | | | | | (SEQ ID NO: 36) |
| Yeast | 71631318 | hsp78 | 41 | 1 | 1 | 1 | AAAAAAAAAAAAAGAAAAUAAAAC |
| | | | | | | | AAAAAAAAAAAAAAAAAAAA |
| | | | | | | | (SEQ ID NO: 37) |

### Example 2 Non-A Modification in Poly(A) Tail Positively Correlates with Translation Efficiency

In this example, the relation between the translation efficiency and non-A modification in poly(A) tail was analyzed using the existing ribosome association data of mouse GV oocytes (GSE 135525), Caenorhabditis Elegans germ cells (GSE 62859) and somatic cells (GSE 58918) and HeLa cells (GSE 79664), and the cumulative curves of translation efficiency of two groups of genes with and without non-A modification in poly(A) tails were compared. The genes under analysis were those containing at least 10 detected poly(A) tails in the datasets. In the data of mouse GV oocytes, a gene “with G” referred to that in the gene data, more than or equal to 25% of poly(A) tails have G; “without G” referred to that in the gene data, less than 25% of poly(A) tails have G; “with C” referred to that in the gene data, more than or equal to 25% of poly(A) tails have C; “without C” referred to that in the gene data, less than 25% of poly(A) tails have C; “with U” referred to that in the gene data, more than or equal to 25% of poly(A) tails have U; and “without U” referred to that in the gene data, less than 25% of poly(A) tails have U. In the data of Caenorhabditis Elegans and HeLa cells, due to the lower sequencing depth of the data and lower content of non-A base than in GV oocytes, a gene “with G” referred to that in the gene data, at least one poly(A) tail has G; “without G” referred to that in the gene data, all poly(A) tails have no G; “with C” referred to that in the gene data, at least one poly(A) tail has C; “without C” referred to that in the gene data, all poly(A) tails have no C; “with U” referred to that in the gene data, at least one poly(A) tail has U; and “without U” referred to that in the gene data, all poly(A) tails have no U.

The modifications of pATIM-G (i.e., poly(A) tail containing G) and pATIM-C(i.e., poly(A) tail containing C) were found to be positively correlated with translational efficiency. Wherein, the translational efficiency (TE) refers to the proportion of mRNA molecules associated with ribosomes for translation in the total mRNA molecules of a particular gene in a sample. The numerical value can be directly calculated by using the data of Ribo-seq and RNA-seq of the gene, and the specific calculation formula is as follows: TE=(FPKM in Ribo-seq)/(FPKM in RNA-seq).

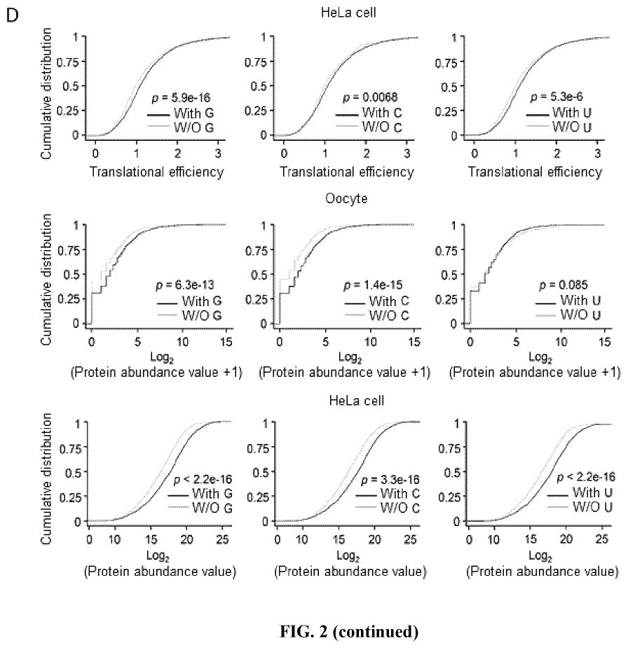

As a result, it was found that pATIM-G and pATIM-C modifications had significantly higher translational efficiencies both in germ cells and in somatic cells (FIG. 2A-D). Besides, PATIM-U (i.e., poly(A) tail containing U) modification can also improve translational efficiency.

Considering that the final result of mRNA translation is the accumulation of the corresponding protein, the inventors analyzed the relationship between non-A modification in poly(A) tail and protein abundance in GV oocytes (i.e., mouse GV oocytes) and HeLa cells using published proteomic data for mass spectrometry to compare cumulative curves of translation efficiency for the two groups of genes with or without non-A modification in poly(A) tails. Published proteomic data for mass spectra were obtained from: Bekker-Jensen et al., 2017; Wang et al., 2010 (10.1016/j.cels.2017.05.009; 10.1073/pnas. 1013185107).

The results show that genes with poly(A) tail containing non-A modifications were expressed in high abundance in both GV oocytes and HeLa cells, which is also consistent with the ribosome binding data (FIG. 2E-F).

The above series of results show that non-A modification in poly(A) tail may play a general regulatory role for mRNA translation in both germ cells and somatic cells. It further demonstrates that poly(A) tail is not only a simple structural component of mRNA, but also can function as a key regulatory factor for mRNA function in germ cells and somatic cells.

### Example 3 GLD-4 enhances translation efficiency by establishing non-A modification in poly(A) tail in Caenorhabditis Elegans

Poly(A) polymerase (PAP) catalyzes the production of RNA poly(A) tails. canonical PAP catalyzes transcription-coupled mRNA polyadenylation, and non-canonical PAP (ncPAP) catalyzes re-polyadenylation of post-transcriptional poly(A) tail (Lim et al., 2014; Lim et al., 2018). Two ncPAPs in Caenorhabditis Elegans, GLD-2 expressed ubiquitously and GLD-4 expressed predominantly in germ cells, are important for the development of germ cells (Schmid et al., 2009; Wang et al., 2002). The knockout of gld-2 can result in a global reduction in the length of poly(A) tails. However, after the knockout of gld-4, there was only a slight decrease in the length of poly(A) tail at transcriptome level (Nousch et al., 2014). The overall translational efficiency in gld-4 mutant decreased, whereas a similar decrease was not observed in gld-2 mutant (Millonigg et al., 2014; Nousch et al., 2014), suggesting that GLD-2 and GLD-4 may have different functions and mechanisms.

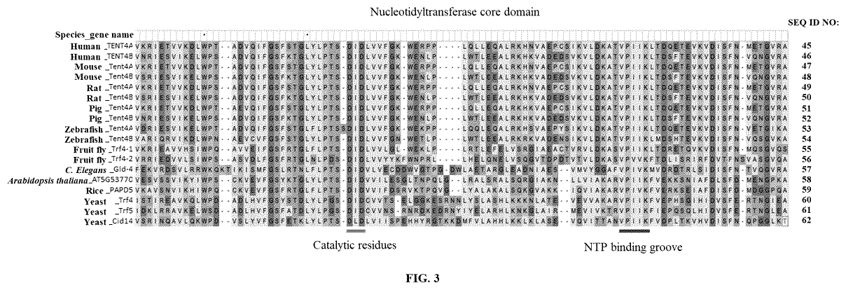

All 10 species mentioned in Example 1 contain 1 or 2 homologous genes of GLD-4, which all encode proteins with conserved core nucleotidyl transferase domains (FIG. 3). The homologous genes of GLD-4 in the mammalian genome are TENT 4A/B. Purified TENT4A and TENT4B proteins can catalyze the production of a poly(A) tail composed primarily of A with simultaneous incorporation of G, U and C. This result is also consistent with the mixed tailing observed in Hela cells (Lim et al, 2018, 10.1126/science. aam 5794).

In this example, it was found that GLD-4, an non-canonical poly(A) polymerase in Caenorhabditis Elegans, regulates translation by establishing non-A modification in poly(A) tail, and GLD-4/TENT4A/TENT4B catalyzes the production of non-A modification in RNA poly(A) tail.

The inventors first extracted total RNA from the gld-4 (ef15) mutant as a test sample, then prepared a PAIso-seq library according to the method of Example 1, carried out third generation sequencing, and analyzed sequences of RNA poly(A) tails. 2 biological replicates were setup in the experiment and wild type Caenorhabditis Elegans (WT) was set as a control. The cDNA sequence of Gld-4 in Caenorhabditis Elegans is shown in SEQ ID NO: 1, which encodes Gld-4 protein shown in SEQ ID NO: 2.

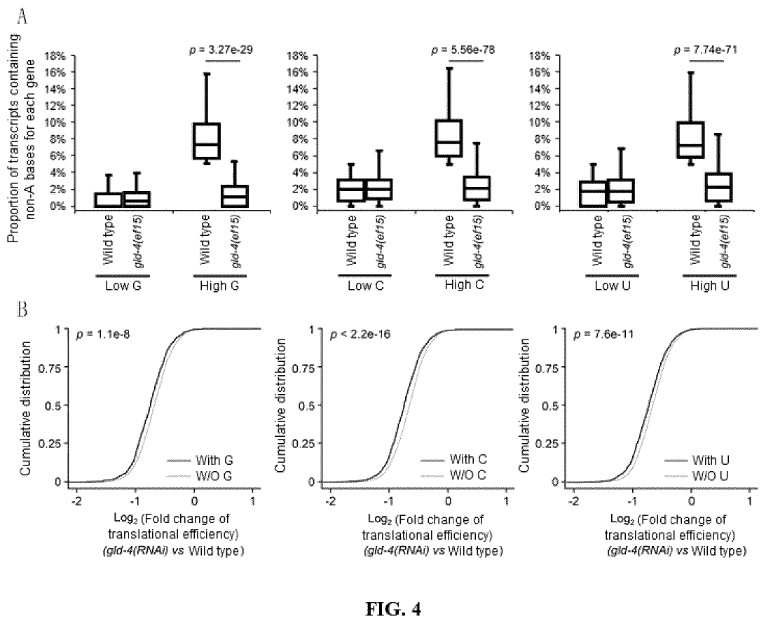

The result shows that in the wild type (WT) transcript, there were genes with poly(A) tail containing non-A modification, while in the gld-4 mutant, there was significant loss of PATIM-G, pATIM-C and pATIM-U modifications (FIG. 4A). This result shows that GLD-4 can catalyze the production of non-A modifications in poly(A) tail of RNA.

Considering that the proportion of transcripts with non-A modification in poly(A) tail in Caenorhabditis Elegans germ cell genes was positively correlated with translational efficiency, the inventors also compared changes in the translational efficiency of genes with and without non-A modification in poly(A) tail before and after the knockout of gld-4.

It was consistent to the expectation that in the gld-4 mutant, genes with non-A modification in poly(A) tail had significantly higher translational efficiency than genes without non-A modification in poly(A) tail (FIG. 4B). This result confirms that GLD-4 can improve translational efficiency by catalyzing non-A modification in poly(A) tail.

In conclusion, ncPAP such as GLD-4/TENT4A/TENT4B can catalyze the production of the evolutionarily conserved non-A modification in poly(A) tail which promotes mRNA translation, thus improving the translational efficiency.

### Example 4 Synthetic Poly(A) Tail Containing Non-A Modification Promotes mRNA Translation

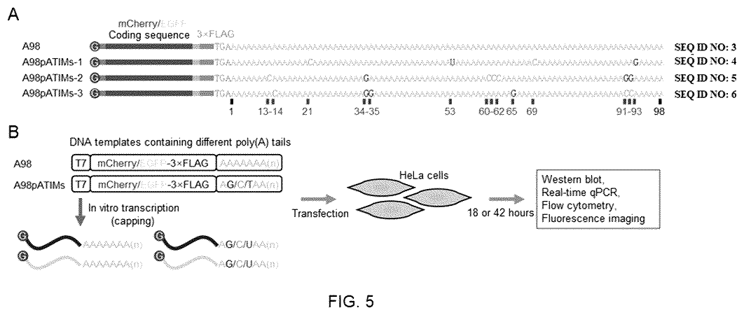

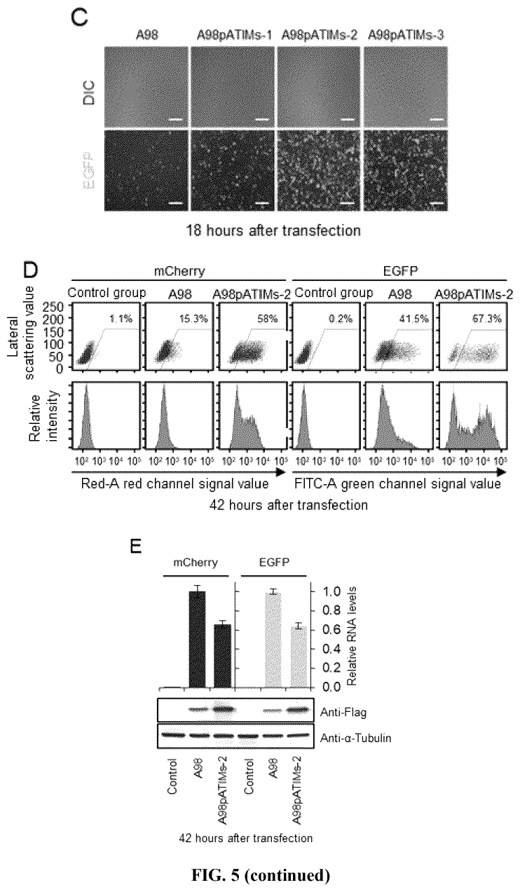

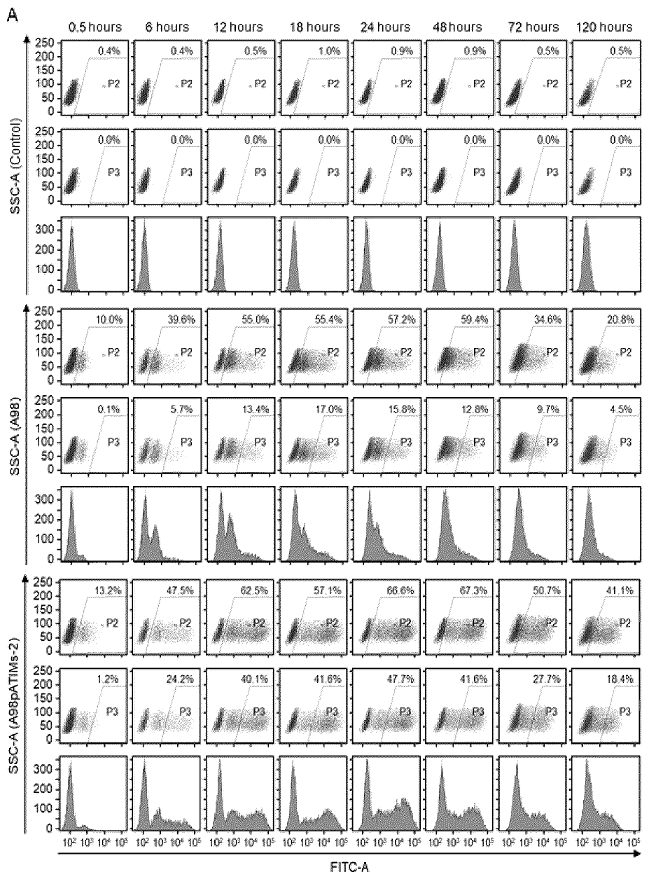

Since non-A modification in poly(A) tail is positively correlated with translational efficiency, and loss of GLD-4 in Caenorhabditis Elegans is accompanied by a decrease in non-A modification of poly(A) tail and a decrease in translational efficiency, the inventors further investigated the role of non-A modification in poly(A) tail in translational efficiency using an in vitro synthesized mRNA reporter system. Fluorescent reporter genes (EGFP and mCherry) mRNA (FIG. 5A) with the same length but different amounts of non-A modification in poly(A) tails were synthesized and then transfected into Hela cells. The dynamic changes of the corresponding reporter mRNAs and proteins were detected (FIG. 5B). The method comprises the following steps:

Four types of EGFP mRNA and four types of mCherry mRNA were synthesized. The four mRNAs of EGFP were mRNAs containing EGFP CDS sequence followed by A98, A98pATIMs-1, A98pATIMs-2 or A98pATIMs-3 sequence, respectively. The four mRNAs of mCherry were mRNAs of containing mCherry CDS sequence followed by A98, A98pATIMs-1, A98pATIMs-2 or A98pATIMs-3 sequence, respectively. Between CDS sequence and A98, A98pATIMs-1, A98pATIMs-2 or A98pATIMs-3 sequence, RNA sequence encoding FLAG tag (GAUUACAAGGACGACGAUGACAAG, SEQ ID NO: 44) was always present.

A98, A98pATIMs-1, A98pATIMs-2 and A98pATIMs-3 are four poly(A) tails, respectively, the sequences of which are set forth in SEQ ID NOs: 3-6 (i.e., the sequences after TGA in FIG. 5A), and A98 does not contain non-A modification in the poly(A) tail. The CDS sequence of EGFP is set forth in SEQ ID NO: 7 (the sequence is from 5′ end to TGA in FIG. 5A), and the CDS sequence of mCherry is set forth in SEQ ID NO: 8 (the sequence is from 5′ end to TGA in FIG. 5A).

Each mRNA was dissolved with RNase-free water and stored at −80° C. for later use.

Hela cells were cultured in a humid incubator with 5% CO2 at 37° C., and the culture medium was DMEM medium (Invitrogen) containing 10% FBS (Gibco). The transfection of HeLa cells with mRNA was performed using Lipofectamine MessengerMAX transfection reagent (Invitrogen). The transfection amount of each mRNA was 2500 ng/(250,000-1,000,000 cells).

At a desired time point after transfection, images were captured, FACS analysis was performed or cells were collected for further experiments.

At 18 hours after transfection, fluorescence signals from cells transfected with different EGFP mRNAs were detected by fluorescence microscopy. The results show that mRNAs with non-A modification in poly(A) tail had significantly higher expression levels of EGFP fluorescent protein compared to the control with poly(A) tail containing A only (A98) (FIG. 5C).

At 42 hours after transfection, the difference between the translation efficiency of the poly(A) tail containing A98pATIMs-2 modification and the poly(A) tail containing A only was compared using FACS. According to the comparison, the EGFP fluorescence of the A98pATIMs-2 reporter gene was enhanced; and a similar fluorescence enhancement was also observed in mCherry (FIG. 5D). These results show that the translation enhancing effect of non-A modification in poly(A) tail is not limited to a specific mRNA. HeLa cells not transfected with mRNA were used as control.

At 42 hours after transfection, protein and mRNA levels in these cells were quantified by immunoblotting and qPCR, respectively. HeLa cells not transfected with mRNA were used as control.

Primers used for qPCR quantification are shown in Table 2.

TABLE 2
Primer sequences

| Gene name | Primer name | Sequence (5'-3') |
|---|---|---|
| mCherry | mCherry-qF | ACGCTGAGGTCAAGACCACCTAC |
| gene | | (SEQ ID NO: 38) |
| | mCherry-qR | TCGTACTGTTCCACGATGGTGTAG |
| | | (SEQ ID NO: 39) |
| EGFP gene | EGFP-qF | GGCACAAGCTGGAGTACAACTACAA |
| | | (SEQ ID NO: 40) |
| | EGFP-qR | GGATCTTGAAGTTCACCTTGATGC |
| | | (SEQ ID NO: 41) |
| Internal | Beta-actin- | TCAGAAGGACTCCTATGTGGGTGAC |
| reference | qF | (SEQ ID NO: 42) |
| gene | Beta-actin- | CCAGTTGGTAACAATGCCATGTTC |
| | qR | (SEQ ID NO: 43) |

Primary antibodies for immunoblotting: murine anti-FLAG antibody (Sigma, F1804; 1:1000 dilution), murine anti-Tubulin antibody (Sigma, T9026; 1:1000 dilution). Secondary antibodies: fluorescent antibody (LI-COR, 925-32210, 1:10000 dilution). Tubulin is an internal reference.

Consistent with the results of fluorescence microscopy and FACS analysis, cells transfected with mRNA having a poly(A) tail containing non-A modification showed higher accumulation of both Cherry and EGFP proteins (FIG. 5E). qPCR results show no increase in the corresponding mRNA levels, indicating that the increase in protein levels was caused by higher translation efficiency rather than stronger mRNA stability (FIG. 5E).

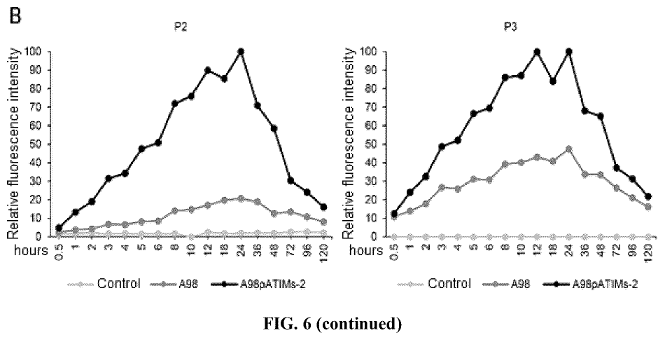

Samples were collected at different time points after transfection for fluorescence detection. The results show that this enhancing effect on mRNA translation was sustained for 6-120 hours after transfection (FIG. 6). HeLa cells not transfected with mRNA were used as control.

The above experiment results clearly reveal that non-A modification in poly(A) tail can greatly promote mRNA translation.

The present invention has been described in detail above. It will be apparent to those skilled in the art that without departing from the spirit and scope of the present invention, the present invention can be implemented within a wide range under equivalent parameters, concentrations, and conditions without undue experimentation. Although the present invention has provided specific examples, it should be appreciated that the present invention may be further modified. In summary, the present invention is intended to include any variations, uses, or modifications of the present invention according to the principles of the present invention, including variations made by known conventional technology in the art which departure from the disclosed scope of the present application. Some of the essential features can be used according to the scope of the claims appended below.

### Industrial Applications

In the present invention, it was found that pATIM is a conserved characteristic of RNA poly(A) tail in eukaryotes and has no species and tissue specificity in distribution through analyzing poly(A) tail in human, mouse, rabbit, pig, zebrafish, fruit fly, Caenorhabditis Elegans, Arabidopsis thaliana, rice and yeast. By analyzing mRNA translation, it was found that pATIM can improve translational efficiency. Genetic analysis showed that GLD-4, an non-canonical poly(A) polymerase in Caenorhabditis Elegans, regulates translation by establishing pATIM. By adding non-A modification in poly(A) tail of mRNA synthesized by in vitro transcription, and comparing the same with the corresponding mRNA containing poly(A) tail of pure A with the same length, it was further found that non-A modification in poly(A) tail can effectively promote mRNA translation, and the addition of G, C and U in poly(A) tail can promote mRNA translation and increase the content of the corresponding protein. This indicates that non-A modification in poly(A) tail in eukaryotes potentially plays a general role of regulating translation, which further demonstrates that non-A modification in poly(A) tail is an effective tool for increasing mRNA translation efficiency.

In recent years, in the field of medical engineering, mRNA-based therapeutic methods have been vigorously developed (Sahin et al., 2014). The cost advantage of mRNA therapy over other protein-based therapies is significant since mRNA is easier to design and prepare. Preclinical development of mRNA vaccines can be very rapid, which often has great potential to cope with emergent new infectious diseases (Deweerdt, 2019; Zhong et al., 2018). Since the outbreak of COVID-19 epidemic, it took only 1 month from the beginning of development of the first mRNA vaccine to the clinical trial at phase 1/2 (Pardi et al., 2018; Thanh Le et al., 2020; Wang et al., 2020). The improvement of mRNA translation efficiency is a major technical challenge in the development of mRNA-based therapeutics and vaccines (Sahin et al., 2014; Zhang et al., 2019). The discovery of a new mechanism for promoting translational efficiency by non-A modification in poly(A) tail can enable the non-A modification incorporated in poly(A) tail of mRNA to be a brand-new technology to further promote various applications of mRNAs such as mRNA-based therapeutics and mRNA vaccines.

1. A method of increasing mRNA translation efficiency comprising applying a poly(A) tail with a non-adenosine (non-A) modification t to a mRNA.

2. (canceled)

3. The method according to claim 1, wherein the non-A modification in the poly(A) tail is selected from the group consisting of insertions of G, C, U and a combination thereof.

4. The method according to claim 1, wherein the mRNA translation is an mRNA translation in a eukaryote.

5. The method according to claim 4, wherein the eukaryote is an animal or a plant or a eukaryotic microorganism.

6. The method according to claim 5, wherein
the animal is a mammal or a non-mammal;
the plant is a dicotyledonous plant or a monocotyledonous plant; and
the eukaryotic microorganism is a yeast.

7. The method according to claim 1, wherein the non-A modification in the poly(A) tail is selected from the group consisting of insertions of G, C, U and a combination thereof; and the mRNA translation is an mRNA translation in a eukaryote.

8. The method according to claim 7, wherein the eukaryote is an animal or a plant or a eukaryotic microorganism.

9. The method according to claim 8, wherein
the animal is a mammal or a non-mammal;
the plant is a dicotyledonous plant or a monocotyledonous plant; and
the eukaryotic microorganism is a yeast.

10. A method of increasing mRNA translation efficiency comprising using a substance that promotes non-A modification in a poly(A) tail.

11. (canceled)

12. The method according to claim 10, wherein the non-A modification in the poly(A) tail is selected from the group consisting of insertions of G, C, U and a combination thereof.

13. The method according to claim 10, wherein the substance that promotes non-A modification in the poly(A) tail is a protein or biomaterial, and
the protein is selected from the group consisting of the following A1)-A5):
A1) a non-canonical PAP;
A2) GLD-4, TENT4A, or TENT4B protein;
A3) GLD-4 protein comprising an amino acid sequence set forth in SEQ ID NO: 2;
A4) a protein which is obtained by substituting and/or deleting and/or adding one or more amino acid residues of the amino acid sequence set forth in SEQ ID NO: 2 in the sequence listing and has the same function; and
A5) a fusion protein obtained by connecting a label at the N end or/and the C end of A1) or A2) or A3) or A4); and
the biomaterial is selected from the group consisting of the following B1) to B5):
B1) a nucleic acid molecule encoding the protein;
B2) an expression cassette comprising the nucleic acid molecule of B1);
B3) a recombinant vector comprising the nucleic acid molecule of B1), or a recombinant vector comprising the expression cassette of B2);
B4) a recombinant microorganism comprising the nucleic acid molecule of B1), or a recombinant microorganism comprising the expression cassette of B2), or a recombinant microorganism comprising the recombinant vector of B3); and
B5) a cell line comprising the nucleic acid molecule of B1), or a cell line comprising the expression cassette of B2).

14. The method according to claim 10, wherein the non-A modification in the poly(A) tail is selected from the group consisting of insertions of G, C, U and a combination thereof;
the substance that promotes non-A modification in the poly(A) tail is a protein or biomaterial, and
the protein is selected from the group consisting of the following A1)-A5):
A1) a non-canonical PAP;
A2) GLD-4, TENT4A, or TENT4B protein;
A3) GLD-4 protein comprising an amino acid sequence set forth in SEQ ID NO: 2;
A4) a protein which is obtained by substituting and/or deleting and/or adding one or more amino acid residues of the amino acid sequence set forth in SEQ ID NO: 2 in the sequence listing and has the same function; and
A5) a fusion protein obtained by connecting a label at the N end or/and the C end of A1) or A2) or A3) or A4); and
the biomaterial is selected from the group consisting of the following B1) to B5):
B1) a nucleic acid molecule encoding the protein;
B2) an expression cassette comprising the nucleic acid molecule of B1);
B3) a recombinant vector comprising the nucleic acid molecule of B1), or a recombinant vector comprising the expression cassette of B2);
B4) a recombinant microorganism comprising the nucleic acid molecule of B1), or a recombinant microorganism comprising the expression cassette of B2), or a recombinant microorganism comprising the recombinant vector of B3); and
B5) a cell line comprising the nucleic acid molecule of B1), or a cell line comprising the expression cassette of B2).

15. The method according to claim 10, wherein the mRNA translation is an mRNA translation in a eukaryote.

16. The method according to claim 15, wherein the eukaryote is an animal or a plant or a eukaryotic microorganism.

17. The method according to claim 16, wherein
the animal is a mammal or a non-mammal;
the plant is a dicotyledonous plant or a monocotyledonous plant; and
the eukaryotic microorganism is a yeast.

18-28. (canceled)

29. A product for increasing mRNA translation efficiency, comprising the substance that promotes non-A modification of a poly(A) tail according to claim 10.


SEQUENCE LISTING (62 sequences)

SEQ ID NO: 1 - DNA, 2538 bp, Caenorhabditis elegans
atgaatgaagacagcagattatcatcatcacaacaaccgtcaacgtcgacacctcgttcatcgattccttcgactatgaattcggatgaaccaaatacgtgtcgccgtcttagtcagtcccaggaacagccatcaacgtccagaacttgcaaaagtgaaactccagagtttggatatagtgattcattgccgttcgccccatggcgaaggaaacggtacggactgaatattcaaggactgcacgaggaaattgtcgatatgtatcattggataaaaccgaacgagatagagtcgcgacttcgaacgaaggtctttgagaaggtccgcgattctgtgttacggcgatggaaacagaaaacaataaaaatctcaatgtttggaagtcttagaacgaatctcttcttacctacttcggatattgacgttctcgttgaatgcgatgattgggttggaacacctggagattggcttgctgaaacagcacgtggacttgaagcggacaatattgcggagtcggtgatggtctatggaggagcatttgtgcctattgtgaaaatggtggatagagatactcgattgagcattgatatttcattcaatacagttcaaggagtacgagcagcttcatacattgcaaaggtgaaagaagagtttccattaatagaaccacttgttctcctgctcaaacaattccttcattaccgtaatctcaatcaaacattcaccggaggtctatcctcctatggactcgtccttttgctcgttaactttttccagctttacgccctaaacatgcgaagtcgaacaatttacgaccgaggagtcaatctaggacacttattgcttcgtttcctcgagctgtattctttagagttcaactttgaagaaatgggaatatcacctggacaatgttgctatattccaaagtcggctagcggagcaagatacggtcacaagcaggctcaaccagggaatctagcattggaagatccattacttactgcaaatgatgtcggtagatctacatacaacttttcttcaatagccaatgcctttgggcaagcttttcaaatattacttgttgccgtcacattacgcgagcgaaaagggaaaaatcatgtggctatgagagcttacaaaggatctcttctccatctcattatgccattcacatcaaaagagctcacatatcgtaactggttaatgagtggtgtgttatcaatgcctggccaagaagcaccggcatcgtatgacttaaatcaattgcataacactttggtttctccgatggtcgatctttcacgatacgcttggcttagaaaagcaccagcgaaggcagaaaagcgggattctcgacctctgacgattgtaaatccagcagatgatcgtcaaactcttgcccaacagttgaagaagcagattctggaacaaactgaagcaaagaaatcgttagaaaaaatgccggcttgtgatgacaacaaaaaagaagaggaattggttgcaacaagagaaactgatgttgaattggaagcagaagatactgaatcagaaggtcatcataatggtgaaaatgatttgattcttactggaccacctctaccaacgtctacacaatccgtcaatacatctgccacagtatctactgcagcgagtatttcggagcgagaagacactgattcaccaggactgtcttcgtcaatgggaaatcaatcttcagaagaagatgaggataacggaattaataatcgaaacaattctgctgttcctgttcaattcaagaaacccttcaatgaagttgttgcccagccagcaagagaatcgaagagaacgcagacaaccagtgaagacaagatgcaagatcaattccatttcaatggatactcatatcctccaccctcacgatatgcagcaggtaccgcggcaccatcacacaaacatcgtaatgcgcacccgcaacggcagcgtccttcaattcgtaatctttcacaaggctctgatgggtcagatgaatacaatgttgaatcatggaacaacaatataagacaaggaagaagagcctcgtcgaattcaccaagtccatccagacagcagacgaatactagaaattgtggacccactaataatattccctacgattcatttcgctcacaaaacaagaattcgacgttagatggttcgaataattcatcagaagagccaataacaatgtatgccgatgttgttaaaaagaagtcttcgattacaacatcaaccaatacttccactgcagatgtcaacgtcaccaatggaaatccaatacccgcaaatggaataattccacaatctatggccgtggtcaacgttggaagaggatcctatagaaatgctcttacaacttcccccatgacgcctccatcagcacatacttcaatgcagaagcaacatcatttgcgtaaagataacgaatgtggtttcgacaacaactcagccacttcttctactgatttaagccatcatcaaccacaattggttccaccggtgaatcgtctgcagcgatag

SEQ ID NO: 2 - protein, 845 aa, Caenorhabditis elegans
MNEDSRLSSSQQPSTSTPRSSIPSTMNSDEPNTCRRLSQSQEQPSTSRTCKSETPEFGYSDSLPFAPWRRKRYGLNIQGLHEEIVDMYHWIKPNEIESRLRTKVFEKVRDSVLRRWKQKTIKISMFGSLRTNLFLPTSDIDVLVECDDWVGTPGDWLAETARGLEADNIAESVMVYGGAFVPIVKMVDRDTRLSIDISFNTVQGVRAASYIAKVKEEFPLIEPLVLLLKQFLHYRNLNQTFTGGLSSYGLVLLLVNFFQLYALNMRSRTIYDRGVNLGHLLLRFLELYSLEFNFEEMGISPGQCCYIPKSASGARYGHKQAQPGNLALEDPLLTANDVGRSTYNFSSIANAFGQAFQILLVAVTLRERKGKNHVAMRAYKGSLLHLIMPFTSKELTYRNWLMSGVLSMPGQEAPASYDLNQLHNTLVSPMVDLSRYAWLRKAPAKAEKRDSRPLTIVNPADDRQTLAQQLKKQILEQTEAKKSLEKMPACDDNKKEEELVATRETDVELEAEDTESEGHHNGENDLILTGPPLPTSTQSVNTSATVSTAASISEREDTDSPGLSSSMGNQSSEEDEDNGINNRNNSAVPVQFKKPFNEVVAQPARESKRTQTTSEDKMQDQFHFNGYSYPPPSRYAAGTAAPSHKHRNAHPQRQRPSIRNLSQGSDGSDEYNVESWNNNIRQGRRASSNSPSPSRQQTNTRNCGPTNNIPYDSFRSQNKNSTLDGSNNSSEEPITMYADVVKKKSSITTSTNTSTADVNVTNGNPIPANGIIPQSMAVVNVGRGSYRNALTTSPMTPPSAHTSMQKQHHLRKDNECGFDNNSATSSTDLSHHQPQLVPPVNRLQR

SEQ ID NO: 3 - RNA, 98 nt, Artificial Sequence
  A98 poly(A) tail
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 98 nt, Artificial Sequence
  A98pATIMs-1 poly(A) tail
aaaaaaaaaaaaaaaaaaaacaaaaaaaaaaaaaaaaaaaaaaaaaaaaaaauaaaaaaaaaaaaaaacaaaaaaaaaaaaaaaaaaaaaaagaaaaa

SEQ ID NO: 5 - RNA, 98 nt, Artificial Sequence
  A98pATIMs-2 poly(A) tail
aaaaaaaaaaaacaaaaaaaaaaaaaaaaaaaagaaaaaaaaaaaaaaaaaaaaaaaaacccaaaaaaaaaaaaaaaaaaaaaaaaaaaaggaaaaaa

SEQ ID NO: 6 - RNA, 98 nt, Artificial Sequence
  A98pATIMs-3 poly(A) tail
aaaaaaaaaaaaacaaaaaaaaaaaaaaaaaaaggaaaaaaaaaaaaaaaaaaaaaaaaaaaaagaaaaaaaaaaaaaaaaaaaaaaaaaccaaaaaa

SEQ ID NO: 7 - RNA, 717 nt, Artificial Sequence
  Sequence encoding EGFP
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaag

SEQ ID NO: 8 - RNA, 708 nt, Artificial Sequence
  Sequence encoding mCherry
auggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaag

SEQ ID NO: 9 - RNA, 95 nt, Homo sapiens
aaaaaaaaaaaaaaaaaaaaaaaaugaaaaaaaaaaaaaaaaaaaaaaaaaaaacaaaaaaaaaaaaacaaaaaaaaaaaaaaaaaaaaaaaaaa

SEQ ID NO: 10 - RNA, 85 nt, Homo sapiens
aaaaaaaaaaaaaaaaaaaaaaaaaaaaaaaaugaaaaaaaaaaaaaaaaaaaaaaaaaaacaaaaaaaaaaaaaaaaaaaaaaa

SEQ ID NO: 11 - RNA, 64 nt, Homo sapiens
aaaaaaaaaaaaaaaaaaaaaacaaaaaaagaauaaaaaaaaaaaaaaaaaaaaaaaaaaaaaa

SEQ ID NO: 12 - RNA, 94 nt, Mus musculus
aaaaaaaaaaaaaaaaaaaaaaaaaaaaaaaaaagaaaaaaacuaaaaaaaaaaaaaaaaaaaaaaaaaaaaaaaaaaaaaaaaaaaaaaaaaa

SEQ ID NO: 13 - RNA, 85 nt, Mus musculus
aaaaaaucaaaaaaaaaaaaaaaaaaaaaaaaaaaagaaaaaaaaaaaaaaaaaaaaaaaaaaaaaaaaaaaaaaaaaaaaaaaa

SEQ ID NO: 14 - RNA, 48 nt, Mus musculus
aaaaaaaacauuuuaaaaaugaaaaaaaaaaaaaacaaaaaaaaaaaa

SEQ ID NO: 15 - RNA, 136 nt, Rattus norvegicus
aaaagcagaaaaaaaaaaaaaaaaaaaaaaaaaaaaaaaaaaaaaaaaaaaaaaaaauaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - RNA, 106 nt, Rattus norvegicus
aaaaaaaaaaaaaaaaaaaaaaaaaaaaauaaaaaaaaaaaaaaaaaaaaaaaaaaaaaaaaaaaaaaaaaaaaaaaaaaaacaaaaaaaaaagaaaaaaaaaaaa

SEQ ID NO: 17 - RNA, 98 nt, Rattus norvegicus
guaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 110 nt, Sus scrofa
aaaaaaaaaaaaaaaaaaaaaaaaaaaaaaaaauaaacaaaaaaaaaaaaaaaaaaaaaagaaaaaaaaaaaaaaaaaaaaaaaaaaaaaaaaaaaaaaaaaaaaaaaaa

SEQ ID NO: 19 - RNA, 59 nt, Sus scrofa
aaaaaaaaaaaaaagaaaaaaaaaaaaaaaaaaaaaaaaaaauaaaacaaaaaaaaaaa

SEQ ID NO: 20 - RNA, 59 nt, Sus scrofa
aaaaaacgucaaaaaaaaaaaaaaaaaaaaaaaaaaaaaaaaaaaaaaaaaaaaaaaaa

SEQ ID NO: 21 - RNA, 160 nt, Danio rerio
aaaaaaaaaaaaaaaaaaaaaaaaaaaaaaaaaaaaaaaaaaaaaaaaaaaaaaaaaaaaaaaaaaaaaaaaaaaaaaaaaaacaaaaaaaaaaaaaaaaaaaaaaaaaaaaaagaaaaaaaaaaaaaaaaaaaaaauaaaaaaaaaaaaaaaaaaaaaa

SEQ ID NO: 22 - RNA, 161 nt, Danio rerio
aaaaaaaaaaaaaaaaaaaaaaaaaaaaaaaaaacaaaaaaaaaaaaaaaaaaaaaaaaaagaaaaaaaaaaaaaaauaaaaaaaaaaacagaaaaaaaaaaaaaaaaaaaaaaaaaaaaaagaaaaaaaaaaaaaaaaaaaaaaaaaaaaaaaaaaaaaa

SEQ ID NO: 23 - RNA, 143 nt, Danio rerio
aaaaaaaaaaaacaaaaaaaaaaaaaaaaaaaaaaaaaaaaaaaaaaaauuaaaaaaaaaagaaaaaaaaaaaaaaaaaaaaaaaaaaaaaaaaaaaaaaaaaaaaaaaaaaaaaaaaaaagaaaaaaaaaaaaaaaaaaaaa

SEQ ID NO: 24 - RNA, 97 nt, Drosophila melanogaster
aaaaaaaaaaagaaaaaaaaaaaaaaaaaaaaaaaaaaaaaaaaaaaaaaaaaaaaaaaaaacaaaaaaaaaaaaaaaaaaaaauaaaaaaaaaaaa

SEQ ID NO: 25 - RNA, 96 nt, Drosophila melanogaster
aaaaaaaaaaaaaauaaaaaaaaagaaaaaaaaaaaaaaaaaaaaaaaaaaaaaaaaaaaaaaaaaaaaaaaaaaaaaaaaaaaaaaaaaaaaaca

SEQ ID NO: 26 - RNA, 63 nt, Drosophila melanogaster
  misc_feature (20)..(20): n is t
  misc_feature (40)..(40): n is t
aaaaaaaaaaaaaaaaaagnaaaaaaaaaaaaaaacaaanaaaaaaaaaaaaaaaaaaaaaaa

SEQ ID NO: 27 - RNA, 87 nt, Caenorhabditis elegans
aaaaaaaaaaaucuuuuuuuugaaaaaaaaaaaaaaaaaaaaaaaaaaaaaaaaaaaaaaaaaaaaaaaaaaaaaaaaaaaaaaaaa

SEQ ID NO: 28 - RNA, 57 nt, Caenorhabditis elegans
aaaaaaaauuauuuuuguuuaaaaacaaaaaaaaaaaaaaaaaaaaaaaaaaaaaaa

SEQ ID NO: 29 - RNA, 96 nt, Arabidopsis thaliana
aaaaaaaaaaaaaaaaaaaaaaaaaaaaaaaaaaaaaaaaaaaaaaaaaaaaaaaaaaaaaaaaaaguaaaaaaaaaaaaaaaaaaaaaaaaaaaa

SEQ ID NO: 30 - RNA, 71 nt, Arabidopsis thaliana
aaaaaaaaaaaaaaaaaaaaaaaaaaaaaauaaaggaaaaaaaaaaaaaaaaaaaaaaaaaaaaaaaaaaa

SEQ ID NO: 31 - RNA, 65 nt, Arabidopsis thaliana
aaaaaaaaaaaaaaaaacaaaaaaaaaaaaaauaaaaaaaaaaaaaaaaaaaaaaaaaaaaaaaa

SEQ ID NO: 32 - RNA, 52 nt, Oryza sativa
aaaaaaaaaaaaaaaaaaaaaaaaaaaaaaaagaaaaaaaaaaaaaaaaaaa

SEQ ID NO: 33 - RNA, 91 nt, Oryza sativa
uuaacaaaaaaaaaaaaaaaaaaaaaaaaaaaaaaaaaaaaaaaaaaaaaaaaaaaaaaaaaaaaaaaaaaaaaaaaaaaaagaaaaaaaa

SEQ ID NO: 34 - RNA, 46 nt, Oryza sativa
aaaaaaaaaaaaaaaaaaaagaaaaaaaaaaaaaaaaaaaaaacaa

SEQ ID NO: 35 - RNA, 56 nt, Saccharomyces cerevisiae
aaaaaaaaaagaaaaaaaaaaaaaaaaaaaaaaucaaaaaaaaaaaaaaaaaaaaa

SEQ ID NO: 36 - RNA, 56 nt, Saccharomyces cerevisiae
aaaagguaaaaaaaaaaaaaaaaaaaaaaaaaaaaaacaaaaaacaaaaaaaaaaa

SEQ ID NO: 37 - RNA, 44 nt, Saccharomyces cerevisiae
aaaaaaaaaaaaagaaaauaaaacaaaaaaaaaaaaaaaaaaaa

SEQ ID NO: 38 - DNA, 23 bp, Artificial Sequence
  Synthetic primer
acgctgaggtcaagaccacctac

SEQ ID NO: 39 - DNA, 24 bp, Artificial Sequence
  Synthetic primer
tcgtactgttccacgatggtgtag

SEQ ID NO: 40 - DNA, 25 bp, Artificial Sequence
  Synthetic primer
ggcacaagctggagtacaactacaa

SEQ ID NO: 41 - DNA, 24 bp, Artificial Sequence
  Synthetic primer
ggatcttgaagttcaccttgatgc

SEQ ID NO: 42 - DNA, 25 bp, Artificial Sequence
  Synthetic primer
tcagaaggactcctatgtgggtgac

SEQ ID NO: 43 - DNA, 24 bp, Artificial Sequence
  Synthetic primer
ccagttggtaacaatgccatgttc

SEQ ID NO: 44 - RNA, 24 nt, Artificial Sequence
  Sequence encoding Flag tag
gauuacaaggacgacgaugacaag

SEQ ID NO: 45 - protein, 99 aa, Homo sapiens
VKRIETVVKDLWPTADVQIFGSFSTGLYLPTSDIDLVVFGKWERPPLQLLEQALRKHNVAEPCSIKVLDKATVPIIKLTDQETEVKVDISFNMETGVRA

SEQ ID NO: 46 - protein, 99 aa, Homo sapiens
VNRIESVIKELWPSADVQIFGSFKTGLYLPTSDIDLVVFGKWENLPLWTLEEALRKHKVADEDSVKVLDKATVPIIKLTDSFTEVKVDISFNVQNGVRA

SEQ ID NO: 47 - protein, 99 aa, Mus musculus; identical to SEQ ID NO: 45

SEQ ID NO: 48 - protein, 100 aa, Mus musculus
VSRIESVIKELWPSADVQIFGSFKTGLYLPTSDIDLVVFGKWENLPLWTLEEALRKIHKVADEDSVKVLDKATVPIIKLTDSFTEVKVDISFNVQNGVRA

SEQ ID NO: 49 - protein, 99 aa, Rattus norvegicus
VKRIETVVKDLWPSADVQIFGSFSTGLYLPTSDIDLVVFGKWERPPLQLLEQALRKHNVAEPCSIKVLDKATVPIIKLTDQETEVKVDISFNMETGVRA

SEQ ID NO: 50 - protein, 99 aa, Rattus norvegicus
VSRIESVIKELWPSADVQIFGSFKTGLYLPTSDIDLVVFGKWENLPLWTLEEALRKHKVADEDSVKVLDKATVPIIKLTDSFTEVKVDISFNVQNGVRA

SEQ ID NO: 51 - protein, 99 aa, Sus scrofa
VKRIETVVKDLWPTAEVQIFGSFSTGLYLPTSDIDLVVFGKWERPPLQLLEQALRKHKVAEPCSIKVLDKATVPIIKLTDQETEVKVDISFNMETGVRA

SEQ ID NO: 52 - protein, 99 aa, Sus scrofa; identical to SEQ ID NO: 46

SEQ ID NO: 53 - protein, 100 aa, Danio rerio
VDRIESVIKELWPTADVQIFGSFSTGLFLPTSSDIDLVVFGKWEKPPLQQLEQALRKHSVAEPYSIKVLDKATVPIIKLTDQETEVKVDISFNVETGIKA

SEQ ID NO: 54 - protein, 99 aa, Danio rerio
VARIQRVIKDLWPNAEVCVFGSFSTGLYLPTSDIDLVVFGNWETLPLWTLEEALRKRKVADENSIKVLDKATVPIIKLMDSHTEVKVDISFNVQSGVKA

SEQ ID NO: 55 - protein, 99 aa, Drosophila melanogaster
VKRIEAVVHSIWPQAVVEIFGSFRTGLFLPTSDIDLVVLGLWEKLPLRTLEFELVSRGIAEACTVRVLDKASVPIIKLTDRETQVKVDISFNMQSGVQS

SEQ ID NO: 56 - protein, 99 aa, Drosophila melanogaster
VRRIEDVVLSIWPSASVDLFGSFRTGLNLPDSIDLVVYYKFWNPRLLHELQNELVQGVTDPDTVTVLDKASVPVVKFTDLISRIRFDVTFNSVASGVQA

SEQ ID NO: 57 - protein, 103 aa, Caenorhabditis elegans
FEKVRDSVLRRWKQKTIKISMFGSLRTNLFLPTSDIDVLVECDDWVGTPGDWLAETARGLEADNIAESVMVYGGAFVPIVKMVDRDTRLSIDISFNTVQGVRA

SEQ ID NO: 58 - protein, 100 aa, Arabidopsis thaliana
VESVSSVIKYIWPSCKVEVFGSYKTGLYLPTSDIDVVILESGLTNPQLGLRALSRALSQRGIAKNLLVIAKARVPIIKFVEKKSNIAFDLSFDMENGPKA

SEQ ID NO: 59 - protein, 100 aa, Oryza sativa
VKAVSNVIKHIWPQCKVEVFGSFRTGLFLPTSDIDVVIFDSRVKTPQVGLYALAKALSQKGVAKKIQVIAKARVPIVKFVERKSEIAFDISFDMDGGPQA

SEQ ID NO: 60 - protein, 101 aa, Saccharomyces cerevisiae
ISTIREAVKQLWPDADLHVFGSYSTDLYLPGSDIDCVVTSELGGKESRNNLYSLASHLKKKNLATEVEVVAKARVPIIKFVEPHSGIHIDVSFERTNGIEA

SEQ ID NO: 61 - protein, 101 aa, Saccharomyces cerevisiae
IDKLRRAVKELWSDADLHVFGSFATDLYLPGSDIDCVVNSRNRDKEDRNYIYELARHLKNKGLAIRMEVIVKTRVPIIKFIEPQSQLHIDVSFERTNGLEA

SEQ ID NO: 62 - protein, 102 aa, Saccharomyces cerevisiae
VSRINQAVLQKWPDVSLYVFGSFETKLYLPTSDLDLVIISPEHHYRGTKKDMFVLAHHLKKLKLASEVQVITTANVPIIKFVDPLTKVHVDISFNQPGGLKT